(19) United States
(12) Patent Application Publication
(10) Pub. No.: US 2011/0184401 A1
(43) Pub. Date: Jul. 28, 2011

(54) CRYOTHERAPY PLANNING DEVICE AND CRYOTHERAPY DEVICE

(76) Inventors: Kansei Iwata, Tokyo (JP); Yasushi Iwata, Tokyo (JP); Taisuke Nagasawa, Tokyo (JP); Masafumi Kawamura, Tokyo (JP); Yotaro Izumi, Tokyo (JP); Masanori Inoue, Tokyo (JP); Norimasa Tsukada, Tokyo (JP); Hideki Yashiro, Kanagawa (JP)

(21) Appl. No.: 13/054,439

(22) PCT Filed: Jul. 10, 2009
(86) PCT No.: PCT/JP2009/062629

§ 371 Date: Apr. 6, 2011

(30) Foreign Application Priority Data
Jul. 15, 2008 (JP) 2008-184264
Oct. 10, 2008 (JP) 2008-263892

(51) Int. Cl. A61B 18/02

(52) U.S. Cl. 606/23

(57) ABSTRACT

The present invention relates to a treatment device utilized in the freezing treatment method and its treatment planning device, and has an object to settle a freezing period·defrosting period according to a size of a treatment portion.

A cryotherapy device comprises a gas supply-exhaust system 100, a control system 200 therefore and a freezing probe system 300. The gas supply-exhaust system 100 supplies a freezing gas and a defrosting gas to a probe 60 of the freezing probe system 300 to freeze and defrost the treatment portion surrounding the tip of the probe 60 by the Joule·Thomson effect. The control system 200 controls the gas supply-exhaust system 100 and makes treatment planning data for this control. The treatment planning data includes a freezing·defrosting sequence to determine the freezing period and the defrosting period. The determination of this sequence is performed by the computer in the control system 200. Further, this sequence is determined corresponding to the focus treatment size according to the freezing·defrosting characteristics of the tissue.

### FIELD OF THE INVENTION

This invention relates to, for performing the extremely low temperature therapy, the cryotherapy planning device, and the cryotherapy device utilizing it.

### BACKGROUND OF THE INVENTION

In recent years, the attention is paid to the cryotherapy which treats a patient's affected portion using extremely low temperature.

This cryotherapy is a treatment that, in the state that a double pipe (coaxial needle) whose tip is acute is applied to the patient's body surface, a long thin introducer is inserted along the central axis of the double pipe and stabs the affected portion; the double pipe is advanced to the affected portion along the introducer and penetrates the affected portion; the coaxial needle may be made to penetrate directly; then, the introducer is extracted, and instead, a freezing terminal (probe) is inserted and loaded along the hollow shaft in the double pipe; a freezing gas (gas and liquid both can be used) and a defrosting gas (gas and liquid both can be used) are supplied; and the affected portion is made necrosis by repeating freezing and defrosting (fusion) the affected portion for a short time.

Applicant filed various patent applications concerning the cryotherapy, and have already been open to public.
Patent document 1: JP, 2007-167100, A
Patent document 2: JP, 2007-167101, A
Patent document 3: JP, 2007-295953, A

### DESCRIPTION OF THE INVENTION

Problem to be solved by the Invention

Conventional freezing therapeutic devices are still not practical technically. For example, a freezing temperature and its freezing time width, and a defrosting temperature and its defrosting time width are settled experimentally. Hereafter, the development of practical and reliable devices having a high safety will be desired.

It is an object of the present invention to provide a cryotherapy planning device and a cryotherapy device which enable a proper freezing therapy based on an analysis result of a freezing·defrosting mechanism in a focus organization.

It is a concrete object of the present invention to provide a cryotherapy planning device and a cryotherapy device wherein a freezing temperature·freezing time width and a defrosting temperature·defrosting time width obtained quantitatively by clarifying and functionizing the characteristic of freezing·defrosting in the focus organization can be utilized for the control of the freezing gas and the defrosting gas.

It is another object of the present invention to provide a cryotherapy planning device and a cryotherapy device which enable safely to purge a freezing gas in an evaporation freezing method utilizing the extremely low temperature liquefied gas as the freezing gas.

### Means for Solving the Problem

The present invention provides a cryotherapy planning device comprising, in a cryotherapy device wherein a freezing and a defrosting can be performed in a freezing period which freezes a treatment portion and a defrosting period in which its defrosting is performed, by a freezing probe having a predetermined section size, that said freezing period and said defrosting period are settled according to an organization and a focus treatment size of the treatment portion.

The present invention provides, further, a cryotherapy planning device comprising, in a cryotherapy device wherein a freezing and a defrosting can be performed in a freezing period which freezes a treatment portion and a defrosting period in which its defrosting is performed, that said freezing period and said defrosting period are settled according to a section size of a freezing probe, and an organization and a focus treatment size of the treatment portion.

The present invention provides, further, a cryotherapy planning device wherein a relation between a kind of diameter of a freezing probe and a focus treatment size, obtained by a diameter size of the freezing probe and a freezing limit size settled according to the diameter size, is memorized as data, and, by using this data, a freezing probe to be used and a focus treatment size are determined.

The present invention provides, further, a cryotherapy planning device wherein a relation between a diameter size of a freezing probe, a freezing limit size settled according to the diameter size and a time width approaching to the freezing limit size, is memorized as data, and, by using this data, a freezing probe to be used, a focus treatment size and a freezing time are determined.

The present invention provides, further, a cryotherapy planning device wherein a relation between a diameter size of a freezing probe, a freezing limit size settled according to the diameter size and a time width approaching to the freezing limit size, is memorized as data, and, by using this data, a freezing probe to be used, a focus treatment size, a freezing time, a defrosting period and/or a repetition of cycle of its freezing and defrosting are determined.

The present invention provides, further, a cryotherapy planning device wherein above relation is controlled by a following equation settled by a diameter of a freezing probe, when y is a freezing size and c is a freezing limit size,

y=a exp(bt)+c

wherein exp is an exponential function, a, b and c are coefficients settled by a treatment portion and a diameter of a probe, being in a relation of a<0, b<0, c>0, and c is the freezing limit size.

The present invention provides, further, a cryotherapy device comprising a freezing probe, a gas control means to send a freezing gas for freezing a treatment portion during a freezing period and to send a defrosting gas for its defrosting during a defrosting period, to the freezing probe, means for setting a freezing probe to be used, a focus treatment size, a freezing time T1 and a defrosting time T2 by utilizing memorized data of a relation between the diameter size of the freezing probe, a freezing limit size settled according to the diameter size and a time width approaching to the freezing limit size, and commanding means for sending a control command to said gas control means so as to freeze at the settled freezing time T1 and to defrost at defrosting time T2.

The present invention provides, further, a cryotherapy device wherein the above relation is controlled by a following equation settled by a diameter of a freezing probe, when y is a freezing size and c is a freezing limit size,

y=a exp(bt)+c

wherein exp is an exponential function, a, b and c are coefficients settled by a treatment portion and a diameter of a probe, being in a relation of a<0, b<0, c>0, and c is the freezing limit size.

The present invention provides, further, in a freezing treatment period planning device which obtains a freezing period T₁ for freezing a treatment portion and a defrosting period T₂ to defrost it by utilizing a freezing probe, a freezing treatment period planning device wherein the freezing period T₁ for freezing the treatment portion surrounding the freezing probe by a freezing gas is settled based on a time t obtained by solving an equation of

y=A ln(t)+B (ln is natural logarithm)

wherein A and B are constants settled by an organization of the treatment portion and y is a focus treatment size and the defrosting period T2 is settled based on a time t obtained by solving an equation of

Z=C−D ln(t)

wherein Z is a defrosting size, and C and D are constants settled by an organization of the treatment portion.

The present invention provides, further, a freezing treatment period planning device wherein, when a freezing period and a defrosting period are made as 1 cycle, the number of a repetition of the cycle is settled.

The present invention provides, further, a freezing treatment period planning device wherein a purge period for purging a freezing gas is added to the freezing period.

The present invention provides, further, a cryotherapy device comprising a freezing probe, a gas control means to send a freezing gas for freezing a treatment portion during a freezing period and to send a defrosting gas for its defrosting during a defrosting period, to the freezing probe, means for setting a freezing period T₁ which is settled based on a time t obtained by solving an equation of

y=A lₙ(t)+B (lₙ is natural logarithm)

wherein A and B are constants settled by an organization of the treatment portion and y is a focus treatment size, and a defrosting period T₂ which is settled based on a time t obtained by solving an equation of

Z=C−D lₙ(t) (lₙ is natural logarithm)

wherein Z is a defrosting size, and C and D are constants settled by an organization of the treatment portion, and commanding means for sending a control command to said gas control means so as to freeze for the settled freezing period T₁ and to defrost for the defrosting period T_{2.}

The present invention provides, further, a cryotherapy device wherein said setting means settle the number of a repetition of cycle, when a freezing period and a defrosting period are made as 1 cycle, and said commanding means send control commands for freezing and defrosting to said gas control means according to the cycle.

The present invention provides, further, a cryotherapy device wherein said setting means add a purge period for purging a freezing gas in case that a liquid freezing gas is utilized to said freezing period, and said commanding means send control commands for freezing, defrosting and purging according to these periods.

Further, the present invention provides a cryotherapy device wherein a gas of a vaporized gas generated in a liquid freezing gas source is used as the purge gas in the purge period.

### Effects of the Invention

According to this invention, since a freezing period and a defrosting period can be determined based on the organization of the treatment portion and the focus treatment size, a proper time and a proper treatment can be realized.

### Best Mode Carrying Out the Invention

A freezing and a defrosting depend on making to the low temperature and making to the high temperature. There are examples for the freezing and the defrosting by using a Joule·Thomson effect and by evaporating a low temperature liquefied gas.

The Joule·Thomson effect is a thermal phenomenon occurred when a predetermined pressure of a gas having a constant temperature, such as room temperature, is suddenly lowered. There are examples to become lower temperature and higher temperature, according to a kind of gas. These are utilized for the freezing and the defrosting. These are realized that, for example, when a probe has a structure so that the gas expands suddenly at the tip thereof, and ordinaly temperature argon gas (Ar gas) and helium (He gas) of high pressure of 30 MP are led to the tip of the probe. Freezing temperature of −125° C. is obtained in Ar gas, and the defrosting temperature of +20° C. is acquired in He gas.

A low temperature liquefied gas is used in evaporation of a liquefied gas. For example, in nitrogen (N2) gas, it liquefies by cooling with the high pressure of 70 MP, and this liquid temperature is about −195° C. On this evaporation, many quantity of heat is taken from the circumference, and makes it freeze. The defrosting is realized by sending out a high-temperature fusion gas (or liquid).

The definition of a focus treatment portion and a focus treatment size is clarified, here.

A part which is treated by one freezing probe in the cycle of one time or multiple times will be called a focus treatment portion, and the size of this focus treatment portion will be called a focus treatment size. The focus treatment portion is as follows.

(1) An example of a focus of small spot size or that they are generated dispersively. In this case, the focus of spot size itself is the focus treatment portion. A size of each focus is the focus treatment size.
(2) A first example of a focus having a big volume or area size. In this case, when the treatment is performed by utilizing a probe of large-diameter size, the whole focus is the focus treatment portion and its size is the focus treatment size.
(3) A second example of a focus having a big volume or area size. In this case, when the focus is segmented continuously and each segment is treated by a probe of small-diameter size, each segment is the focus treatment portion and its size is the focus treatment size.

The inventor of this application has discovered the relation between a freezing temperature and its freezing size. It is described below.

When freezing a certain focus treatment portion, a freezing is performed over a certain time with a certain freezing temperature. However, if a freezing temperature is settled, the maximum freezing size is settled with this freezing temperature, and the freezing size does not expand beyond it even though the freezing time is extended. And in the intermediate freezing time until it reaches the maximum freezing size, the freezing size is settled with a certain functional relation to the freezing time.

This is applied to the spot freezing source. The spot freezing source is a freezing source of a spot size, and, in this invention, the tip of the freezing probe serves as this spot freezing source. If the diameter size of the spot freezing source is as extremely small as being disregarded, the freezing size δ becomes

[Numerical Formula 1]

δ≦δ max.

Here, the freezing size δ is settled according to the length of the freezing time.

When the diameter size r₀ of the freezing probe is taken into consideration, it becomes

[Numerical Formula 2]

δ−r₀≦δ max.

The relation between the focus treatment size to be treated and the freezing temperature of the freezing probe is that the focus treatment size needs to be equal or smaller than the maximum freezing size δ max settled by its freezing temperature. This maximum freezing size δ max is the constant c which is settled by the numerical formula 4 described later.

The defrosting is the reverse of the freezing and, fundamentally, the same view as the freezing can be applied.

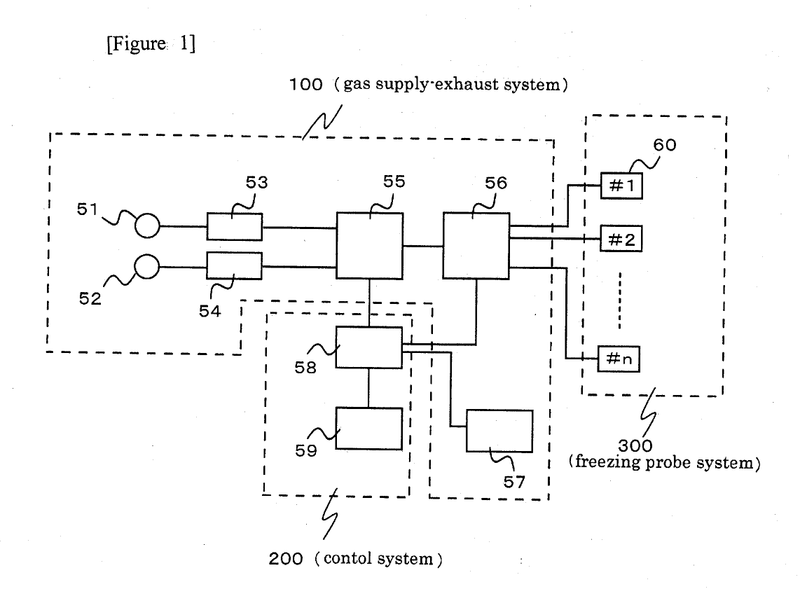

FIG. 1 is a general view of an embodiment of a cryotherapy device of the present invention using the Joule·Thomson effect. This treatment device comprises a gas supply-exhaust system 100, a control system 200 and a freezing probe system 300.

The gas supply-exhaust system 100 comprises a source 51 of gas (for example argon gas) of a room temperature with a high pressure, a source 52 of gas (for example helium gas) of a room temperature with a high pressure, gas stabilizers 53 and 54, a gas switching·pressure gauge portion 55, a distribution·changeover portion 56 and a gas exhaust control portion 57.

The source 51 of high pressure gas functions as a gas source for freezing by making extremely low temperature (in argon, about −125° C.) based on the Joule·Thomson effect, and the source 52 of high pressure gas functions for defrosting from the freezing state by making high temperature (in helium, about +25° C.) based on the Joule·Thomson effect.

The gas stabilizers 53 and 54 are for making the pressure of the high pressure gas from the gas source 51 and 52 constant.

The gas switching·pressure gauge portion 55 is the portion containing a switch for changing a passage of gases from the gas sources 51 and 52, an electromagnetic valve and a branching pipe as the switching means, and a various kinds of monitoring instruments.

The distribution·changeover portion 56 is the distributing and changing means for selecting a probe to be used from the probes 60 having a plurality of probes and for selecting gas supplied to it.

The gas exhaust control portion 57 is the means for exhausting the used gas from the probes 60, and includes the purging means in a treatment device utilizing the evaporation phenomenon wherein the liquid gas is used for freezing, described later.

The control system 200 comprises the control·measurement portion 58 and the control computer 59.

The control computer 59 has a various kinds of data and the treatment programs, and performs the instruction and the monitor of the treatment execution. The treatment execution is attained by controlling the gas switching and pressure gauge portion 55, the gas distribution·changeover portion 56 and the exhaust portion 57. The control computer 59 makes control commands required for those controls, and sends to the control·measurement portion 58.

The control·measurement portion 58 generates a control signal to each portion 55, 56 and 57 according to the control command from the control computer 59, and controls the specific treatment means. The control·measurement portion 58, further, takes measured signals being data and status data of various instruments in each portion 55, 56 and 58, and monitors the control and the operation. For example, the monitoring data is transmitted through the distribution and changeover portion from sensors, such as a thermo couple equipped in the probe, and is taken into the computer 59.

In addition, the control computer 59 has various data containing the patient's ID information, the treatment history, the name of a disease, the treatment portion and its position including the name of an organ, the focus size, physiological data including the blood pressure and the blood sugar level, etc., and the photographic image data of the affected portion, etc.

The treatment program is a software including the execution process of cryotherapy and is formed with the treatment planning and said various data.

The freezing system 300 has plural probes (#1˜#n) 60. The probes 60 are the same form or, also the different form each other, and there are examples of using one and of using plural (2 or 3 pieces) concurrently. These numbers and the usages are included in the treatment program. The size of the diameter size, the length of the probe, the quantity of the gas, and various contents are called the form here. The wall of the probe is equipped with the thermo couple, and the temperature of the probe is sent to the control-measurement portion.

The treatment program has the contents of processing containing the treatment procedure, the freezing·defrosting sequence (the number n of the cycle, when the freezing and defrosting are made as 1 cycle, the time width T of the 1 cycle), the risk management of the treatment, the monitor management of the various kinds of living body watching equipments and devices including X-ray CT device, the electrocardio device and the manometer, etc for monitoring the treatment execution and watching.

The negative pressure system about the exhaust is explained.

High pressure gas is sent through the distribution and changeover mechanism to the probe 60 in the probe system 300, expands in the probe according to the Joule-Thomson effect, and is discharged through the exhaust system in a negative pressure. In this exhaust system, the negative pressure is held by the negative pressure generating mechanism installed in the exhaust control system 57, and prompt discharge is carried out. A negative pressure monitoring sensor is equipped in the negative pressure generating mechanism, and an output of this sensor is sent to the computer 59. The computer 59 watches the negative pressure. If negative pressure turns it to positive, the probe will be filled with a sending-out high pressure gas, and will result in a very dangerous state. When it is judged that this negative pressure changed to positive pressure or high positive pressure, the computer 59 urgently stops the control system.

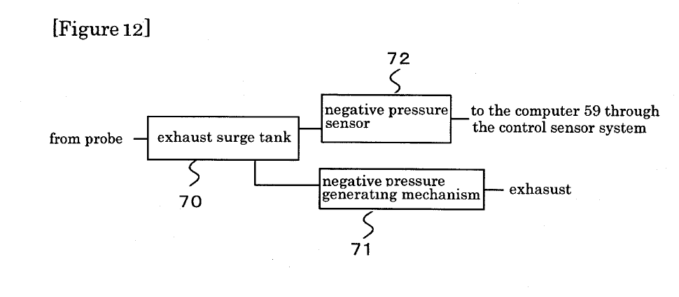

Such embodiment is shown in FIG. 12. This exhausting system has an exhaust surge tank 70, a negative pressure generating mechanism 71 and a negative pressure sensor 72. The exhaust gas from an exhaust path of the probe goes into the exhaust surge tank 70. The exhaust surge tank 70 is maintained at a negative pressure by the negative pressure generating mechanism 71. The negative pressure sensor 72 detects that a return exhaust system of the probe is kept in a negative pressure and is in an aspiration state. The output of the sensor is always inputted into the computer 59 and watched that it is normal. When it changes into the contrary state, the control system 57 is immediately stopped and the sending-out of the high pressured gas is stopped.

In the treatment program, the freezing·defrosting sequence is especially concerned with this present invention. In the freezing·defrosting sequence, the number n of the cycle is usually a value of more than 1, and can be changed variously, such as n=2 or n=3. The time width T of the cycle can also be changed variously. The time width T of 1 cycle is the total value of the freezing time width (freezing period) T₁ and the defrosting time width (defrosting period) T2. The width T₂ corresponds to the time width necessary for defrosting the freezing in the width T₁.

The number n of cycle and the time width T of 1 cycle are decided with the treatment size (diameter, volume or cross sectional area) of the focus of the freezing object. When the focus treatment size is large, at least either one of n and/or T is settled large. For example, in case n=3, it is adjusted by T; when T is fixed, n is made large or small according to the size. There is also an example wherein both of n and T are changed.

Hereinafter, the many features, functions and effects of the present invention are explained, in the treatment program execution, by limiting to the execution of freezing·defrosting sequence. In FIG. 1, it is also shown that only the composition along with such meaning.

First, it is described that the determination method of 1 cycle time width T according to the dimension of the focus treatment size of the freezing treatment portion in case that the number of cycle n is n=3.

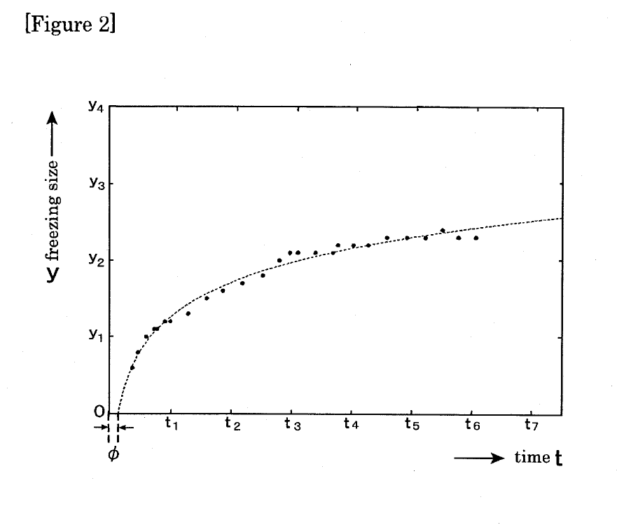

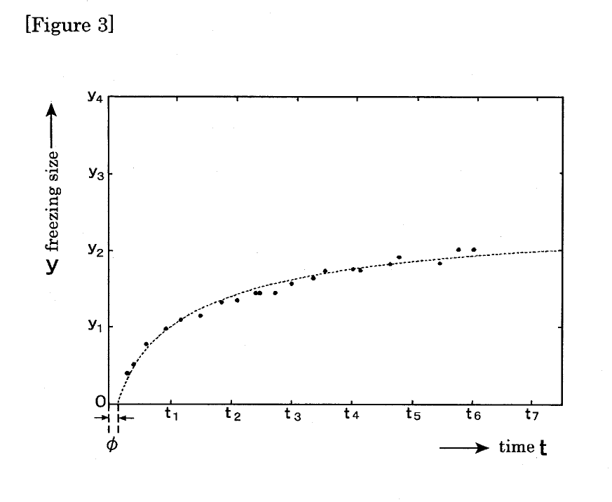

When the focus treatment size is large, the large freezing energy is required. This freezing energy depends on the length of 1 cycle time T. The applicant of this application searched for the relation between the focus treatment size and the freezing time by the animal experiment under the use of freezing probe whose section size (a diameter size in a circle, and an area size is also included) is fixed. FIG. 2 shows an example of freezing in a lung, and FIG. 3 shows an example of freezing in a liver. The transverse designates the freezing time t and the vertical axis designates the freezing size (diameter) y generated by the freezing. The freezing is also called the congelation. Because the freezing size y corresponds to the freezing temperature AT (extremely low temperature, such as around −125° C.), the vertical axis may be designated according to the scale of the freezing temperature AT. For example, the freezing size y is values from several millimeters to several ten millimeters, and the freezing time t is values of from several ten seconds to several hundreds of seconds.

The example of approximation functions of plotted points in FIGS. 2 and 3 becomes, in the method of least squares, a following formula.

[Numerical Formula 3]

y=A lₙ (t)+B.

Here, it is designated that A and B are values mostly settled with the kind (organization), the state and the size of a region of organ, such as a lung and a liver, the freezing temperature depending on the freezing gas to be used, etc., and the diameter of the probe, and lₙ is a natural logarithm. The time Φ(=exp (−B/A)) at the start of the function in FIGS. 2 and 3 corresponds to the diameter of the probe.

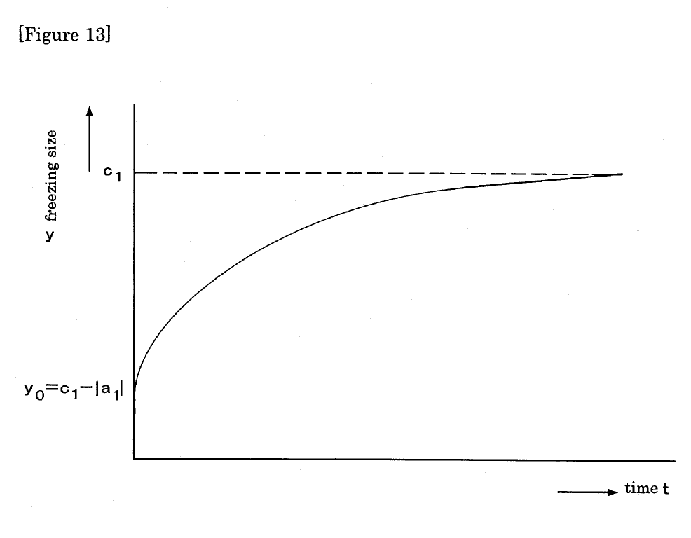

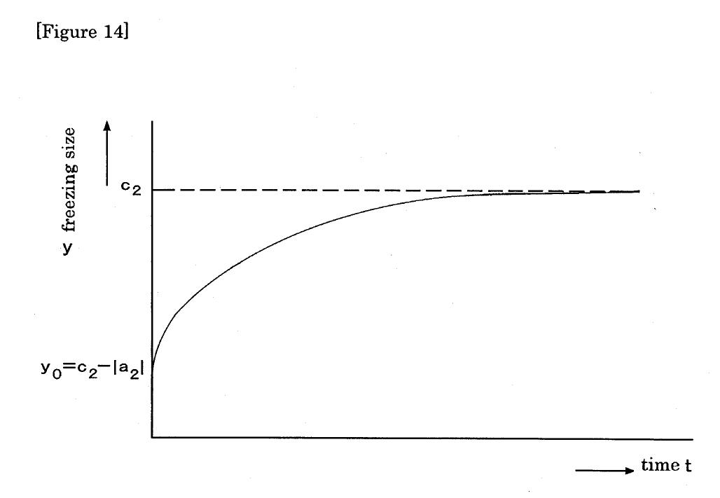

FIGS. 13 and 14 are the examples expressed FIGS. 2 and 3 on another scale. However, the experimental data (dots) are omitted in FIGS. 13 and 14. Figures which shifted FIGS. 2 and 3 to left and enlarged time enough are FIGS. 13 and 14. The intersections y=y₀ (c₁−|a₁ |) and (c₂−|a₂|) of y-axes at the transverses t=0 show the diameters of the probes. That is, the diameter y₀ of the probe was made into the initial value of the freezing. To enlarge time enough means here to have taken the time beyond the time for the freezing limit which is that the freezing size cannot advance anymore.

The example of approximation functions based on the method of least squares of FIGS. 13 and 14 becomes a following formula.

[Numerical Formula 4]

y=a exp (bt)+c.

Here, it is designated that a, b and c are values mostly settled with the kind (organization), the state and the size of a region of organ, such as a lung and a liver, the freezing temperature depending on the freezing gas to be used, etc., and the diameter of the probe, and are a<0, b<0 and c>0.

That is, coefficients A, B, a, b, and c are considered to be uniquely decided according to the size of the diameter of the probe when the kind and the state of the focus treatment and the freezing temperature are settled.

Differences between the numerical formula 3 and the numerical formula 4 are as follows;

(1) The numerical formula 4 is a formula in consideration of the freezing limit size. The freezing limit size is the maximum size of the freezing area generated, when the probe stabbed the focus, around the stabbed portion. When a focus organ is specified, the freezing limit size is decided by the diameter of the probe. The larger the diameter becomes, the larger its size becomes; and the smaller the diameter, the smaller its size.

The value of y becomes y=c at t=∞ in the numerical formula 4. This value c is the freezing limit size. In practice, y mostly saturates in a limited and short time width, such as 3 or 7 minutes, instead of t=∞, and becomes to the freezing limit size c (c₁ in FIG. 13 and c₂ in FIG. 14). Therefore, the freezing time width for a long time is unnecessary.

In the numerical formula 4, the value c+a of y at t=0 shows the diameter of the probe. Since a<0, c −|a| becomes the diameter of the probe.

(2) In the numerical formula 3, y=∞ at t=∞, on the expression, and is not saturated. Therefore, it is not employable for the freezing time of the time width which is saturated. On the contrary, it is used for determining the freezing time wherein the freezing is not saturated.

Next, each meaning and utilization of the numerical formula 3 and the numerical formula 4 is explained.

(1) There is a meaning that the numerical formula 3 is applicable to the determination of the freezing time for the freezing size which does not reach to the freezing limit size. The example of use is described later.

(2) There is a meaning that the numerical formula 4 is applicable to carry out a freezing of the size near to the freezing limit size. Specifically, it becomes a view as follows.

i. There is a meaning that a proper treatment is enabled. Since the treatment size is made to correspond to the freezing limit size, the proper treatment of only the focus, without damaging the normal organization around the treatment focus, can be attained. Further, since the freezing limit size is obtained by saturation in 3 or 5 minutes, the freezing time for a long time is unnecessary and the rapid treatment can be attained.
ii. There is meaning that the freezing probe having a proper diameter can be chosen. The freezing limit size is basically determined by the diameter of the freezing probe. Therefore, when the treatment size is settled, the freezing probe having the diameter corresponding to it can be chosen, and more suitable treatment can be taken. For example, the diameter sizes of the probe are various, such as 1 mm, 2 mm and 3 mm. When the freezing limit size of each diameter size described above is c₁, c₂ and c₃, the probe of 1 mm is chosen for the focus treatment size of c₁, and the probe of 2 mm is chosen for c₂. There may be the case that not accord correctly. If it is middle size of c₁ and c₂, for example, 1 mm sized can be used in 2 steps overlapping a partial area.
iii. There is an example that the freezing size is made below the freezing limit size.

At that time, the freezing limit size is a criterion for the moment, the freezing time (period) is chosen so as to become the freezing size below it.

iv. It is described how to decide the freezing time t.

When making it freeze to the size near the freezing limit size c, the time width is selected so as to almost reach the value c (that is, to reach the saturation state on the curve). When setting it as the size not close to the freezing limit size c, there is also a method to solve the numerical formula 4 alike FIG. 3.

Utilizations of the numerical formula 3 and the numerical formula 4 are explained.

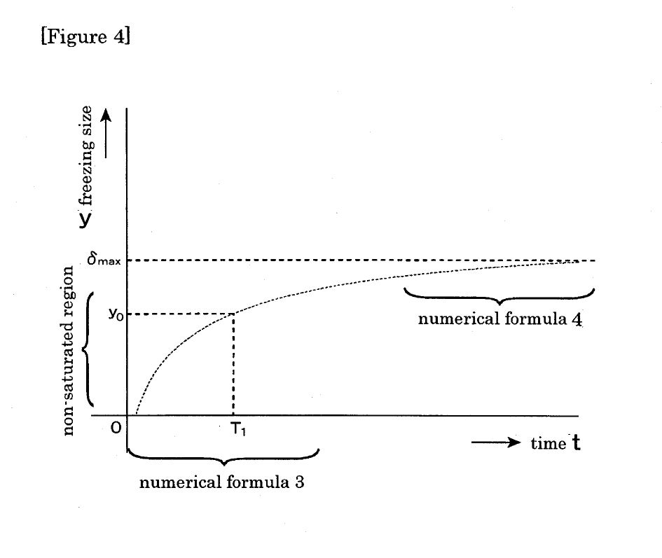

Since, in the numerical formula 3, the focus size is also the target freezing size y₀, values A, B and y=y₀ become fixed values, and the time t can be found by solving the numerical formula 3. On the other hand, when making it freeze to the size near the freezing limit size c, utility time to reach saturation is found by the numerical formula 4. This found time t is equivalent to the freezing time width T₁. FIG. 4 shows this relation for simulation. δ max is the maximum limit freezing size c, and is the value in the saturation state on the curve. The defrosting time T₂ is also calculated by the similar view.

The experimental data in FIG. 2, FIG. 3, FIG. 13 and FIG. 14 change also with the kind of the freezing gas, the sending-out speed of the freezing gas and the diameter of the probe. The various values of A and B are asked based on the kind of freezing gas, its sending-out speed, the diameter of the probe and the organ portion, and stored in the memory of the computer 59; at the occasion of the treatment, the corresponding and applicable values of A and B are read out, the focus size y is inputted, and the time width t is asked by the numerical formula 3.

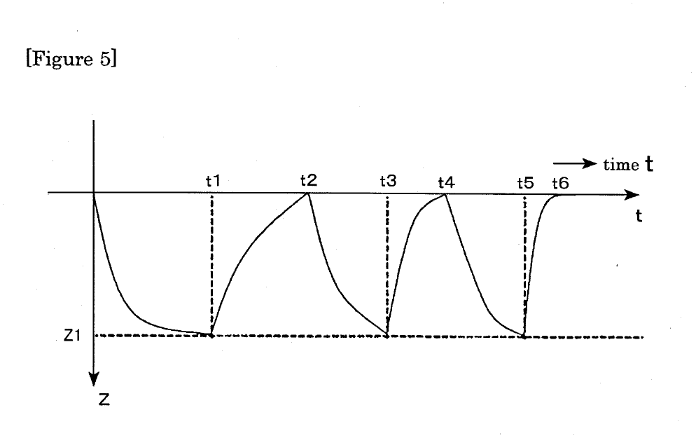

An example of freezing·defrosting sequences is shown in FIG. 5. A transverse shows the time t and a vertical axis shows the freezing size Z. Z₁ is the maximum freezing size (it is not the maximum limit in the example of use of the numerical formula 3, and it is near the maximum limit size in the example of use of the numerical formula 4). The sequence shown in this Figure is as follows;

0˜t₁ . . . 1^{st} freezing section

t₁˜t₂ . . . 1^{st} defrosting section

t₂˜t₃ . . . 2^{nd} freezing section

t₃˜t₄ . . . 2^{nd} defrosting section

t₄˜t₅ . . . 3^{rd} freezing section

t₅˜t₆ . . . 3^{rd} defrosting section.

0˜t₂ is the 1st cycle, t₂˜t₄ is the 2^{nd} cycle and t₅˜t₆ is the 3^{rd} cycle. In the Figure, the cycle widths was made as 1^{st} cycle>2^{nd} cycle>3^{rd} cycle. This is because that the effect of freezing is noticeable in 1^{st} freezing-defrosting and the freezing effect can be continued with the energy less than that in 2^{nd} and 3^{rd}. Obviously, there is also an example having the same time width.

In the example using the numerical formula 3, the defrosting is in a reverse relation to the numerical formula 3, such as the numerical formula 5, for example.

[Numerical Formula 5]

Z=C−D lₙ (t)

Even in this expression, C and D are values obtained in advance as of A and B (usually C=B, D=A) and Z is the defrosting size (this is also the freezing size), and, by solving the numerical formula 5. time t, i.e., the aforementioned T₂ is obtained. Since values C and D change also with the inflow speed of the defrosting gas, C and D are settled based on these various parameters (the focus portion, the inflow speed, the kind of defrosting gas) and stored in the memory. They are read out at the time of the determination of the treatment sequence and the defrosting time width is decided according to the defrosting size.

A necrosis of a focus treatment portion depends on the freezing time width T₁ and the number n of the cycle, the freezing gas, and the inflow speed of the freezing gas. Especially, since the cycle number n is the frequency in which the freezing and the defrosting are repeated, there are many examples that the necrosis is difficult in only 1 time of the cycle and there are many examples that the complete necrosis is accomplished by using 2 cycles˜5 cycles. When the number of the cycle becomes large, the treatment time becomes long and hence the pains of the patient becomes great, and when the number of the cycle becomes small, it becomes difficult to accomplish the complete necrosis. The proper number n of the cycle is selected so as to mutually compensate the such shortages.

The number of the cycle is explained further, here.

The number n of the cycle is selected so as to accomplish the treatment effect without the patient's pains. The patient's pain is that the treatment time becomes long, and the treatment effect is that the focus treatment portion can be necrotized. In the values more than n=1, n=2˜5 was the practical value. Although the treatment time became long compared with n=1, the effect of necrosis was fully accomplished. This was confirmed by the experiments of Kansei Iwata, et al., the inventors of this application. The example of n=2 is explained, hereinafter.

The freezing of the 1st time is a treatment for the focus treatment portion being the living body tissue (this is the living body tissues, regardless to tumor or normal cell, as a group of cells surrounding the air chamber in lung), and the freezing of the 2ⁿd time is for the defrosting result of the 1^{st} time. In the freezing of the 1^{st} time for freezing the living body tissue, a frozen body (ice block) and a portion in semi-frozen state in the outside of its circumference appear. This portion of the semi-frozen state is a big enlarged area as compared with the body. The outside of the semi-frozen state portion is in a normal living body state.

When the defrosting is performed to this freezing, the frozen body liquefies and the semi-frozen state portion also liquefies according to it. In such a liquefied portion, it is simultaneously accompanied by bleeding.

In the freezing of the 2^{nd} time, the frozen body in a strong liquefaction freezes promptly and the semi-frozen state part in a weak liquefaction also freezes successively. That is, the freezing of 1^{st} time was mainly for the freezing of the frozen body, in the 2^{nd} freezing, enlarged semi-frozen state portion surrounding it is also frozen. Thus, the necrosis of the living body tissue is performed including the semi-frozen state portion. The defrosting is carried out after the completion of the freezing.

In the 2^{nd} time, the semi-frozen state may be disregarded and the freezing function of the same coefficient as the 1^{st} time may be used. However, in taking a semi-frozen state into consideration, the coefficient is settled considering the freezing to the frozen body and also to the semi-frozen state. In this case, when n=2 in treatment planning by the example using the numerical formula 3, it is settled the coefficients A, B, C and D according to each cycle, and it is preferable to settle the focus treatment portion to the size of the semi-frozen state enlarged in the 1^{st} cycle.

Of course, there is an example that the necrosis effect is also accomplished with the example of n=1. Further, n=3˜5 is an example that the 3^{rd}˜5^{th} cycle accomplishes the further necrosis effect.

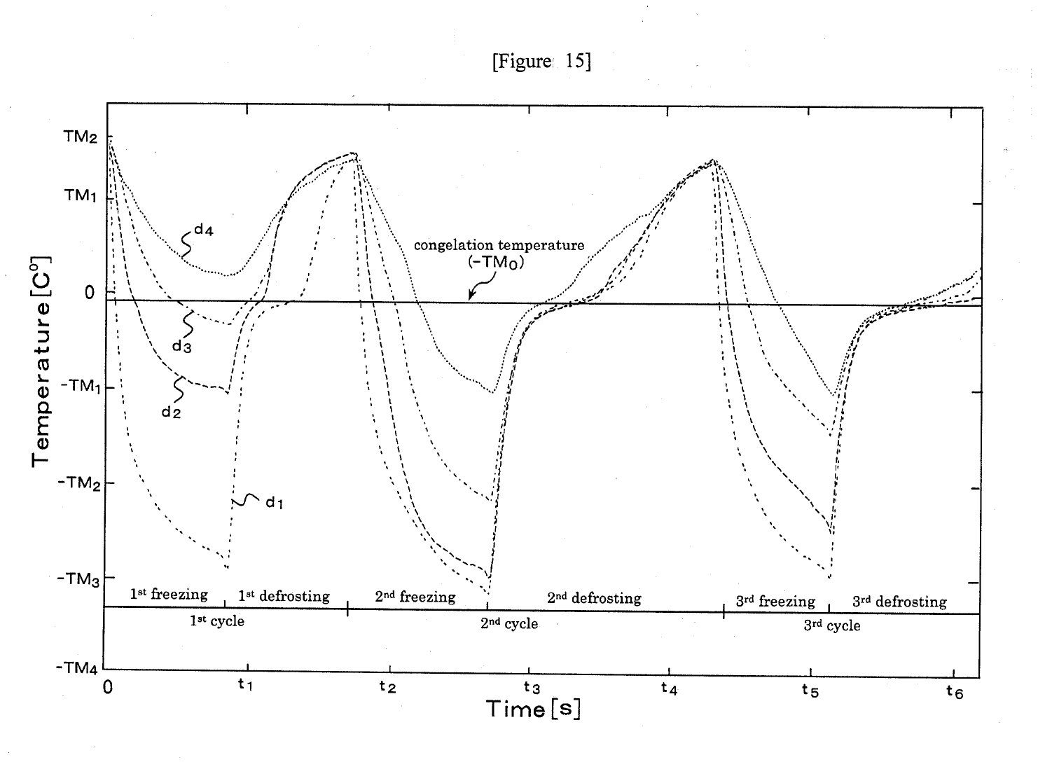

FIG. 15 is an explanatory view of the frozen body and the semi-frozen state around it. This figure is the example figure for confirmation of the experiments, in the example of 3 cycles, of the temperature TM and the time t at a position d of each portion on a concentric circle from a freezing center. The position d of the each portion shows the diameter on the 4 concentric circles having the relation of d₁<d₂<d₃<d₄, such as d₁=4 mm, d₂=6 mm, d₃=8 mm and d₄=10 mm, for example. In FIG. 15, the temperature TM measured on each point of the 4 concentric circles in the process of 3 cycles is shown. Though the temperature is TM₁=20° C., TM₂=40° C., −TM₁=−20° C., −TM₂=−40° C., −TM₃=−60° C. and −TM₄=−80° C., the diameter and the temperature are mere one example.

The points recognized according to FIG. 15 are enumerated.

(1) Temperature—TM₀ being a little lower than 0° C. was regarded as the congelation temperature.

(2) The interval of the 2^{nd} cycle is longer than that of the 1^{st} cycle.

(3) Smaller diameter d₁ freezes promptly, and it reaches the temperature −TM₂ in the 1^{st} cycle. Larger diameter d₄ freezes late. It does not freeze in the 1^{st} cycle but freezes, for the first time, in the 2^{nd} cycle. In the 3^{rd} cycle, the freezing temperature further becomes low. The diameters d₂ and d₃ carry out the intermediate behaviour.

(4) Thus, it is understood from the FIG. 15 that the nearer to the freezing center the freezing progresses promptly, the farther it takes a long time for freezing. The portion frozen in the 1^{st} cycle is the aforementioned freezing body, the non-frozen portion existing in its circumference becomes in the semi frozen state. The frozen mass in the 2^{nd} cycle is larger than that of the 1^{st} cycle, and it sometimes becomes more than double.

(5) In addition, the treatment portion is fundamentally maintained in a temperature of, usually, the patient's body temperature (36° C. etc. variously). The normal temperature differs variously according to the region of the body, such as relatively high in the region near the artery. Therefore, the freezing and the defrosting conditions change according to these regions and, so, each coefficient of the freezing function and the defrosting function also takes various values.

Operation of an embodiment of FIG. 1 is explained.

(1) Generation Of Treatment Planning Data Including Treatment Sequence (Freezing·Defrosting Sequence).

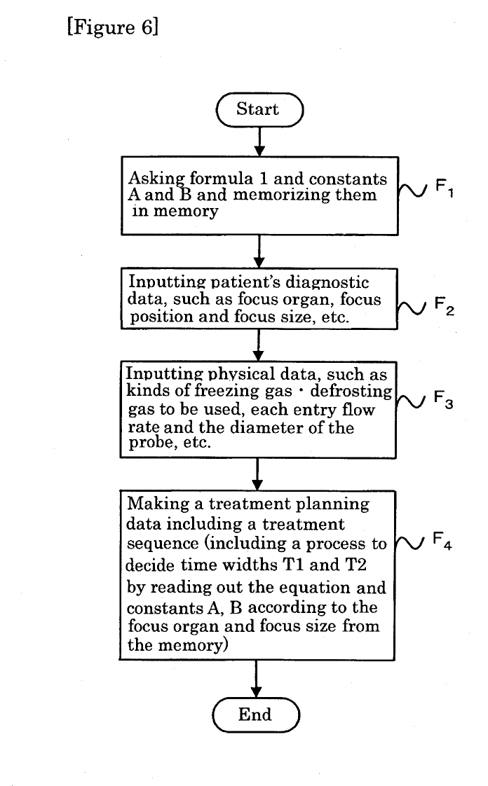

The generation flow of the treatment planning data utilizing the numerical formula 3 is shown in FIG. 6. In the flow F₁, the equation of the numerical formula 3 and the constants A, B (C and D are also included) are asked according to the organs, such as lung and liver, the diameter of the probe and a kind of gas, etc., and stored in the memory of the computer 59. In the flow F₂, patient's diagnostic data (a focus organ, a focus position, a focus size and a classification of focus, etc.) is inputted.

In the flow F₃, the physical data of each mechanical system for a treatment, such as kinds of the freezing gas and the defrosting gas to be used, each entry flow rate and the diameter of the probe, etc. is inputted. In the flow F₄, the treatment planning data including the treatment sequence is made by using the data of the flows F₂ and F₃ and reading out A, B and the equation etc. from the memory. This treatment sequence is, in a narrow sense, freezing·defrosting sequence wherein the freezing·defrosting is made as 1 cycle, and comprises the number n of cycle, the freezing time width T₁ and the defrosting time width T₂. T₁ and T₂ are obtained by the numerical formula 3 and the numerical formula 5. The number n is settled by an experimental value or according to T₁ and T₂. It is, in a broad sense, includes the treatment process before and after the freezing·defrosting sequence. For example, it includes each work of the initialization and the attachment, etc. of various kinds of monitoring apparatus (a display, an electrocardiograph, an X-ray apparatus, etc.) before entering into the freezing·defrosting sequence.

As other treatment planning data, it is included that the treatment procedure data from the start to the end of the treatment, the data of notes in the treatment (for example, the other organ exists in near), and the operating procedure data of the gas supply system (51˜56) and the exhaust system (57) for the freezing·defrosting which is a part thereof.

(2) Execution Of The Treatment

The treatment is performed based on said generated treatment planning data. Although the flow of the whole treatment is omitted, the one concerning to this invention is execution of the freezing·defrosting sequence.

According to the shift to the freezing·defrosting sequence from the computer 59, the freezing treatment is carried out by the inflow of the freezing gas, in case of the freezing, through 51→53→55→56→60 (more than 1 or 2 among them) by means of the control portion 58. In case of the defrosting, by the inflow of the defrosting gas through 52→54→55→56→60 (more than 1 or 2) by means of the control portion 58, the defrosting is carried out.

Various methods for execution of the treatment are shown.

(1) There is a method of mostly full automation by attaching the manipulator to the probe.

(2) The manipulator is attached to the probe, and a person for the operation operates the probe through the manipulator and controls the inflow and the outflow of gas according to the procedure displayed on a screen corresponding to the treatment sequence. In this method, the treatment sequence only displays the operation data which needs for the treatment operation on the screen, and hence the person for the operation becomes to treat according to the operation data.

(3) There is also a method which is interim between abovementioned (1) and (2), i.e., the method wherein a part is automated and a part is operated on manual.

(4) The example by the numerical formula 4 is explained.

In the example by the numerical formula 4, when the treatment portion is settled and the treatment size is settled, the probe having the freezing limit size corresponding to the treatment size is selected. Of course, it is also premised to settle the kind of gas to be used. The freezing cycle is settled in consideration of the treatment effect. Others are the same as that of the numerical formula 3.

Other embodiment is explained.

In the cryotherapy device wherein the freezing is performed by making the liquefied gas, for example, liquid nitrogen gas (for example −195° C.) flow in, the liquefied gas for freezing may remain in the probe at the time of defrosting. Since there is a possibility of evaporating at a stretch and exploding when the defrosting gas (including liquid) is flowed in this state, it is difficult to realize the cryotherapy device wherein the liquefied gas is flowed in. Then, the embodiment which solves such a problem is shown below. This embodiment is that the purge period for purging the liquefied gas is employed in the freezing period, and the vaporized gas of the liquefied gas concerned is utilized for the purge.

Hereafter, the embodiment of this viewpoint is explained.

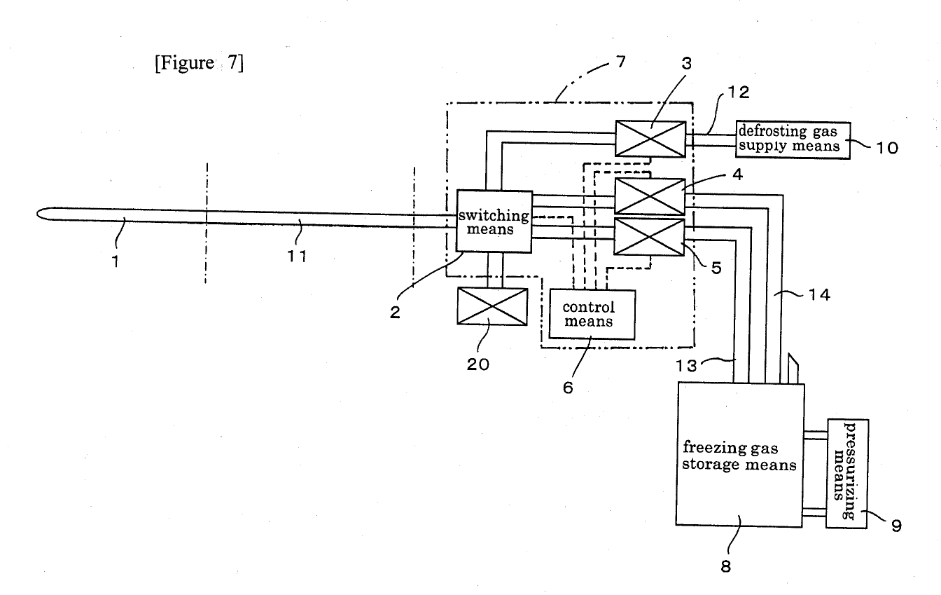

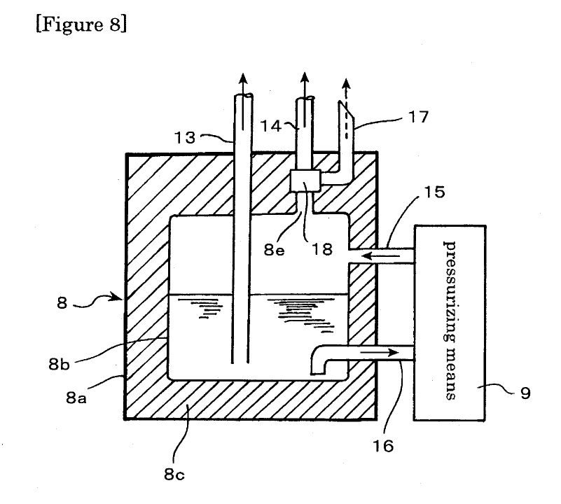

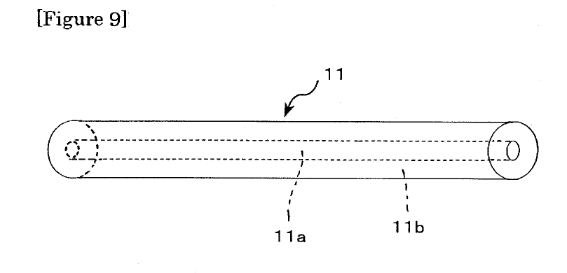

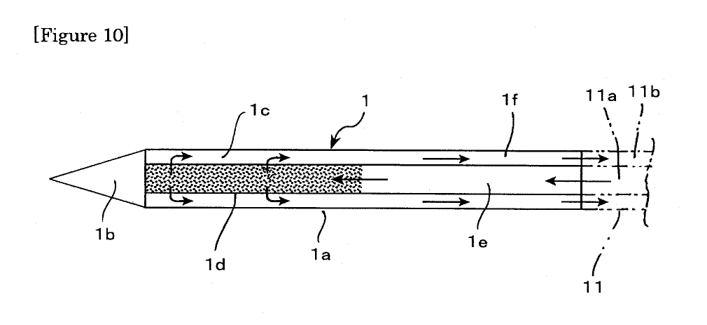

FIG. 7 is a structural sample view of the mechanical system of the cryotherapy device, i.e., the gas supply-exhaust system 100 and the probe system 300 (example of 1 probe), FIG. 8 is an enlarged sectional view of a liquefied gas storage means, FIG. 9 is a perspective view of the gas supply-exhaust pipe connecting the cryotherapy device and the probe, and FIG. 10 is an enlarged sectional view of the probe.

The cryotherapy device shown in FIG. 7 comprises the probe 1 composing of the freezing treatment apparatus, the gas switching control means 7 composing of the gas switching means 2 which supply freezing gas and defrosting gas alternatively to the probe 1 in the treatment and control means 6 which controls the gas switching means 2 and plural opening-shutting valves 3, 4 and 5, the freezing gas storage means 8 used as the freezing gas supply source and connected to the gas switching control means 7, the pressurizing means 9 which pressurizes the inside of the freezing gas storage means 8 at a predetermined pressure, and the defrosting gas supply means 10 connected to the 1st switching valve 3 of the gas switching control means 7, and the gas switching control means 7 is connected to the probe 1 by the gas supply-exhaust pipe 11 which has a flexibility.

This cryotherapy device is a mechanical system, and, although not illustrated, it is controlled by the control section 58 of the computer etc. shown in FIG. 1.

The control is realized by control of the interactive man-machine method through the screen according to the directions of the operator (the person for the operation). The main points of the control are the freezing·defrosting sequences being the treatment processing and are explained later.

The probe 1 composing of the freezing treatment apparatus consists of, as shown in FIG. 8, the probe body 1a and the stabbing portion 1b which forms the tip portion of the probe 1. The probe body 1a is formed by the stainless steel pipe of 2 mm˜3 mm in the outer diameter and the evaporation chamber 1c is located in the tip side of the probe body 1a. The evaporation means 1d for evaporating the liquefied freezing gas is formed in the central portion of the evaporation chamber 1c.

The evaporation means 1d comprises the perforated pipe having a plurality of small through-holes, from which the liquefied freezing gas spouts into the chamber 1c, in the peripheral wall of the thin stainless steel pipe of about 0.6 mm in the outer diameter, for example, and is set the central portion of the chamber 1c so as to be parallel to the axis of the probe body 1a. Its one end side reaches the stabbing portion 1b of the probe body 1a and the other end side is connected to the one end side of the gas outward path 1e provided on the base end side of the probe body 1a.

The gas outward path 1e consists of, as same as the evaporation means 1d, the thin stainless steel pipe of about 0.6 mm in the outer diameter, and is set the central portion of the probe body 1a so as to be parallel to the axis and to provide the freezing gas and the defrosting gas to the perforated pipe 1d. The gas return path 1f for exhausting the gas spouted from the evaporation means 1d to the evaporation chamber 1c is formed between the outer surface of the gas outward path 1e and the inner surface of the probe body 1a.

The other end sides of the gas outward path 1e and the gas return path if are connected respectively to the one end sides of the gas outward path 11a and the gas return path 11b of the gas supply-exhaust pipe 11 connected to the base end side of the probe body 1a.

The gas supply-exhaust pipe 11 has the double pipe structure, as shown in FIG. 9, of the inner pipe 11c and the outer pipe lid consisting of an elastic flexible pipe, so that it may not become the hindrance of the operation to stab the probe 1 to the affected portion.

The inside of the inner pipe 11c is the gas outward path 11a and the path between the inner pipe 11c and the outer pipe lid is the gas return path 1b. The other end side of the gas supply-exhaust pipe 6 is connected to the gas switching means 2 provided to the gas switching control means 7.

The gas switching control means 7 comprises, as shown in FIG. 7, the 1^{st}, 2^{nd} and 3^{rd} opening-shutting valves 3, 4 and 5 consisting of a plurality of electromagnetic valves, the gas switching means 2 consisting of the multi-way valve for supplying gas, which is supplied selectively by the 1^{st}, 2^{nd} and 3^{rd} opening-shutting valves 3, 4 and 5, to the probe body la through the gas supply-exhaust pipe 11, and the control means 6 for controlling the switching of the 1^{st}, 2^{nd} and 3^{rd} opening-shutting valves 3, 4 and 5 and the gas switching means 2.

Opening and shutting control of the 1^{st}, 2^{nd} and 3^{rd} opening-shutting valves 3, 4 and 5 and the gas switching means 2 are carried out by the switching operation timing beforehand programmed in the control means 6, and, to the 1^{st} switching valve 3 of the gas switching control means 7, the defrosting gas supply means 10 to supply the defrosting gas, such as the helium gas, is connected through the defrosting gas supply pipe 12.

On the other hand, to the 2^{nd} switching valve 4 and the 3^{rd} switching valve 5, the freezing gas storage means 8 is connected.

The freezing gas storage means 8 accommodates, as shown in FIG. 8, the storage tank 8b of airtight structure wherein the freezing gas (for example CO2 liquefied gas) is stored in a box-like case 8a. The thermal insulator 8c is filled between the case 8a and the storage tank 8b, and the inside of the storage tank 8b is always maintained at a predetermined temperature.

In the storage tank 8b, there is the liquefied freezing gas of about ½ of full capacity in the lower part thereof, and the vaporized freezing gas of about ½ of full capacity in the upper part thereof.

The gas of the freezing gas stored in the upper part of the storage tank 8b has the temperature of mostly same as that of the liquefied freezing gas. This freezing gas (hereinafter called a purge gas) is utilized as a purge gas for discharging the freezing gas and the defrosting gas stagnated in the probe body 1a.

The freezing gas feed pipe 13 and the purge gas feed pipe 14 are set in the upper part of the storage tank 8b.

The lower end of the freezing gas feed pipe 13 reaches near the bottom of the storage tank 8b and is immersed in the portion of the liquefaction of the freezing gas so as to supply only the liquefied defrosting gas. The lower end of the purge gas feed pipe 14 is connected to the opening 8e on the upper surface of the storage tank 8b so that only the purge gas can be supplied.

The pressurizing means 9 is connected to the storage tank 8b, and the inside of the storage tank 8b is always pressurized in the predetermined pressure.

The pressurizing means 9 consists of, for example, a pump, and is connected to the lower portion of the storage tank 8b via the suction pipe 16 to inhale the freezing gas stored in the lower portion of the storage tank 8b. The freezing gas pressurized at the predetermined pressure by the pressurizing means 9 is exhaled to the purge gas stored in the upper portion of the storage tank 8b via the discharge pipe 15.

It is set in the purge gas feed pipe 14 that the safety valve 18 for preventing the inside of the storage tank 8b from becoming higher than the upper limit pressure by the discharge of the purge gas in the storage tank 8b to the air via the exhaust pipe 17 when the pressure in the storage tank 8b reaches the upper limit pressure settled beforehand.

The numeral 20 in FIG. 7 is the exhaust means consisting of the exhaust valve to discharge the freezing gas, the defrosting gas and the purge gas to the air, and is connected to the gas switching means 2.

Now, the concrete method in the case of the treatment of a malignant tumor is explained utilizing the cryotherapy devices of the embodiments shown in FIG. 7˜FIG. 10.

First, the probe 1 is inserted into the inside of the patient's body so that the tip of the probe 1 of the freezing treatment apparatus arrives at the malignant tumor tissue, and the tip portion of the probe 1 is made to penetrate to the patient's affected portion.

Next, the gas switching control means 7 is switched to the freezing treatment mode, and the freezing treatment is started. After the 1^{st} switching valve 3 is closed, the 2^{nd} switching valve 4 is opened and the 3^{rd} switching valve 5 is closed according to the operation timing beforehand programmed in the control means 6, the gas switching means 2 is switched in the direction so that the 2^{nd} switching valve 4 and the probe 1 becomes in the open state each other. Hence, the purge gas stored in the state of pressurized to, for example, 150 kg/cm2 at the maximum by the pressurizing means 9, in the upper portion of the storage tank 8b is supplied to the probe 1 through the gas outward path 11a of the gas supply-exhaust pipe 11 and the purge process is carried out.

The purge gas supplied to the probe 1 reaches the perforated pipe ld through the gas outward path 1e in the probe body 1a, is exhausted into the evaporation chamber 1c through the small through-holes provided in the peripheral wall of the perforated pipe 1d, reaches the gas switching means 2 through the gas return path 1f in the probe body 1a and the gas return path 11b of the gas supply-exhaust pipe 11, and is exhausted to the air from the exhaust means 20 connected to the gas switching means 2.

Thus, the air remained in the gas supply-exhaust pipe 11 and the probe 1 is purged, and the purge process of the air is completed.

Next, after the 1st and 2nd switching valves 3 and 4 are closed, and the 3rd switching valve 5 is opened by the control means 6, the gas switching means 2 is switched in the direction so that the 3rd switching valve 5 and the probe 1 becomes in the open state each other. Hence, the liquefied freezing gas stored in the state of pressurized to, for example, 150 kg/cm2 at the maximum by the pressurizing means 9, in the lower portion of the storage tank 8b is supplied to the probe 1 through the gas outward path 11a of the gas supply-exhaust pipe 11 and the freezing process is carried out.

The freezing gas supplied to the probe 1 reaches the evaporation means 1d consisting of the perforated pipe through the gas outward path le in the probe body 1a, and is spouted in misty state into the evaporation chamber 1c through the small through-holes provided in the peripheral wall of the perforated pipe 1d. Since it is evaporated in the evaporation chamber 1c, the surrounding heat is taken away by the evaporation heat, the probe 1d is cooled, and hence the freezing of the affected portion is started.

The freezing gas evaporated in the evaporation chamber 1c reaches the gas switching means 2 through the gas return path 11b of the gas supply-exhaust pipe 11, and is exhausted through the gas switching means 2 to the air from the exhaust means 20.

After the freezing process is completed by the progress of the time programmed beforehand, the freezing process is switched to the defrosting process. In the transition period from the freezing process to the defrosting period, the purge process of freezing gas is carried out.

That is, after the freezing process of the affected portion is completed, the control means 6 makes the 1st switching valve 3 in close, 2^{nd} switching valves 4 in open and the 3^{rd} switching valve 5 in close, and switches the gas switching means 2 in the direction so that the 2^{nd} switching valve 4 and the probe 1 becomes in the open state each other.

Thus the purge gas stored in the upper part of the storage tank 8b is supplied to the probe 1 through the gas outward path 11a of the gas supply-exhaust pipe 11, and the purge gas supplied to the probe 1 reaches the evaporation means 1d through the gas outward path 1e, and is exhausted into the evaporation chamber 1c through the small through-holes provided in the peripheral wall of the evaporation means 1d.

Further, since it reaches the gas switching means 2 through the gas return path if in the probe body 1a and the gas return path 11b of the gas supply-exhaust pipe 11 and is exhausted to the air through the exhaust means 20 from the gas switching means 2, the remained gas, which is not evaporated, in the perforated pipe 1d of the probe 1 and the liquefied freezing gas remained in the evaporation chamber 1c are exhausted with the purge gas to the air through the exhaust means 20, and hence all the freezing gas remained in the probe is exhausted.

Since the freezing gas vaporized in the storage tank 8b is used for the purge gas for discharge the liquefied freezing gas remained in the probe 1, the purge gas has almost same temperature as that of the liquefied freezing gas and thus the freezing gas remained in the probe 1 is discharged without changing the temperature in the probe 1.

After the purge process is completed by the discharge of the remained freezing gas in the probe 1, it is changed to the defrosting process. The 1st switching valve 3 is changed to open, the 2^{nd} and 3^{rd} switching valves 4 and 5 are changed to close and then the gas switching means 2 is switched in the direction so that the 1^{st} switching valve 3 and the probe 1 becomes in the open state each other by the control means 6. The defrosting gas such as helium is supplied to the probe 1 through the gas outward path 11a of the gas supply-exhaust pipe 11 from the defrosting gas supply means 10 connected to the 1^{st} switching valve 3, and hence the defrosting process is carried out.

The defrosting gas supplied to the probe 1 reaches the perforated pipe 1d through the gas outward path le in the probe body 1a, is vaporized in the evaporation chamber 1c by the spout in misty state into the evaporation chamber 1c through the small through-holes provided in the peripheral wall of the perforated pipe 1d, and the defrosting of the affected portion frozen by the freezing gas is started.

The defrosting gas evaporated in the evaporation chamber 1c reaches the gas switching means 2 through the gas return path 1f in the probe body 1a and the gas return path 11b in the gas supply-exhaust pipe 11, and is exhausted to the air from the gas switching means 2 through the exhaust means 20.

The input amount of heat added to the affected portion by the freezing gas is calculated by the theoretical formula, and it is required, for defrosting the frozen affected portion by the defrosting gas, to apply the amount of heat equivalent to that of the freezing, by the defrosting gas, to the affected portion. It is omitted here as to the theoretical formula.

After the defrosting process programmed aforehand is completed, the defrosing process is changed to the purge process, and then the purge process is carried out again.

By the freezing process and the defrosting process are alternately repeated by repeating supply of the freezing gas, the purge gas and the defrosting gas alternately, the malignant tumor tissue of the affected portion is necrotized and the treatment effect by the cryotherapy comes to be acquired.

Since the freezing and the defrosting of the affected portion is carried out, in a short time, efficiently by putting the purge process between the freezing process and the defrosting process, the treatment time becomes short and hence the patient's pain is reduced.

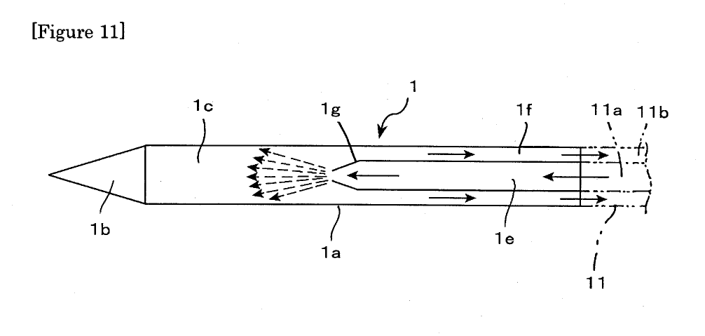

Though, in the above-mentioned embodiments, the evaporation means 1d consisting of the perforated pipe is provided in the anterior portion of the probe body 1a, the liquefied freezing gas is vaporized by spouting in misty state into the evaporation chamber 1c through the small through-holes provided in the peripheral wall of the perforated pipe, and the affected portion is frozen by the evaporation heat generated at this time, it may be made, as shown in FIG. 11, that the evaporation means 1g consisting of the nozzle is provided in the anterior portion of the probe body 1a, the liquefied freezing gas is vaporized by spouting in misty state into the evaporation chamber 1c from this evaporation means 1g, and the affected portion is frozen by the evaporation heat generated at this time.

Although the unnecessary freezing gas, purge gas and defrosting gas were discharged from the exhaust means 20 to the air, it may be made to return the freezing gas and the purge gas to the storage tank 8b, and the defrosting gas to the defrosting gas supply means 10.

Although the probe 1 stabbed directly the patient's affected portion in the freezing treatment, it may be made that an introducer beforehand stabs the affected portion and then the probe 1 stabs the patient's affected portion using the introducer as the guide. Also it may be made that a outer sheath pipe stabs the patient's affected portion using the introducer as the guide; the introducer is drawn out from the outer sheath pipe when the tip of the outer sheath pipe penetrates the affected portion; in this state, the probe 1 is inserted into the outer sheath pipe and stabs the affected portion; and after the outer sheath pipe is extracted, the freezing and the defrosting of the affected portion is carried out by the probe 1.

Although the example of the cryotherapy method for the malignant tumor utilizing the probe was explained, it can be applied to overall cryotherapy devices which are used for the diseases that the cryotherapy method is effective.

The purge period is a short time compared with the freezing time width and the defrosting time width, and hence it is rare to affect the freezing and the defrosting. However, in case that the purge period is taken in consideration, it should be considered that, in the purge period of the freezing gas, for example, how it gives the freezing the influence, and that, in the purge period of the defrosting gas, how it gives the defrosting the influence. Its degree of incidence can be settled variously to the values defined experientially, and to the values settled in consideration of the freezing energy and the defrosting energy, etc.

There is the following way, for example.

In explaining the cryotherapy method which treats the affected portion by utilizing said constituted cryotherapy device, the treatment sequence is explained first.

The sequence of the treatment process is the processes repeating plural cycles (such as 2 times or 5 times), when the freezing process time T₁ and the defrosting process time T₂ is made as 1 cycle. There are both cases, T₁=T₂ and T₁≠T₂. T₁ and T₂ may change for every cycle. For example, times in 2^{nd} and 3^{rd} cycles are made shorter than T₁ and T₂ in the 1^{st} cycle. This is because that the freezing and the defrosting are performed in the 1^{st} cycle and hence lesser energy of the freezing and the corresponding lesser energy of the defrosting are sufficient after the 2^{nd} cycle.

The freezing process time T₁ is the time width for freezing. Specifically, it is the total value of the period for freezing actually (actual freezing period) T₁₁ by opening the 3^{rd} switching valve 5 and hence carrying out the freezing gas to the tip in the probe 1, and the purge period T₁₂ for purging compulsorily the freezing gas to the outside from the inside of the probe 1.

The defrosting process time T₂, which starts at the end of the purge, is the total value of the period for defrosting actually (actual defrosting period) T₂₁ by opening the 1^{st} switching valve 3 and hence performing the defrosting gas to the tip of the probe 1, and the purge period T₂₂ for purging the defrosting gas compulsorily to the outside from the inside of the probe 1.

Although the freezing in the freezing process time T₁ is basically settled by the real freezing period T₁₁, the freezing continues by the influence of the freezing gas remained during the purge, since it is difficult to purge the freezing gas immediately even in the purge period T₁₂.

Therefore, in order to realize the freezing effect, it is necessary to consider the T₁₁ and T₁₂ in series. Then the above-mentioned numerical formula 3 is used.

On the other hand, the freezing gas is decreased gradually by the purge during the purge period and hence the freezing ability becomes small. Therefore, it only has to consider the degree of influence to the freezing size in the purge period in consideration of the decrease of the freezing ability. There is a view as follows.

(1) There is a way that the purge period T₁₂ which is fixed by the purge speed and the total amount of the freezing gas is settled; the freezing size y₀which grows in the period T₁₂ is obtained experimentally or theoretically; and these are added to the numerical formula 2. That is,

[Numerical Formula 6]

y=A ln (t)−B+y₀.

In this formula, t is obtained by the replacement of y with the focus size S₀ for the treatment. This t obtained is the period T₁₁.

(2) There is a way that the quantity of the freezing gas remained in the probe is set to C; the quantity of the purge gas carried out in a unit time is set to D; and these are added to the numerical formula 6. That is, when y=S₀, the time width t is obtained following formula

[Numerical Formula 7]

y=A ln (t)−B+(C/D)·t.

This time width t is (T₁₁+T₁₂). The allotment of T11 and T12 is decided by the proportional distribution of {A ln (t)−B} and (C/D)·t.

(3) In the actual treatment, there is also an example which is not y=S₀. In this case, it is solved by that the relation between y and S₀ is asked in advance and S₀ under that relation is replaced by y. It is also the way to solve by that the S₀ is settled larger than the freezing size y, such as S₀=k y (now, k>0). Further, there is the example that S₀ is settled so as to include the doubtful portion near the circumference of the focus by settling S₀ larger than the actual focus size.

(4) It is also the same in the examples of the numerical formula 4.

Although the numerical formula 3 and the numerical formula 5 are the approximation of the natural logarithmic function and the numerical formula 4 is the approximation of the exponential function, it does not adhere to the natural logarithmic function when it has, as a result of the statistical regression analysis, a higher degree of regression analysis than the natural logarithmic function. There is also function expression with high order of approximation by the fixed time variable, and this is not barred, either.

### BRIEF DESCRIPTION OF THE DRAWINGS

[FIG. 1] It is the figure of whole embodiment of the cryotherapy device of this invention.

[FIG. 2] It is the example of the animal experimental data.

[FIG. 3] It is the example of the animal experimental data.

[FIG. 4] It is the figure of the example of the function expression of the animal experimental data.

[FIG. 5] It is the figure of the example of the freezing·defrosting sequence of this invention.

[FIG. 6] It is the figure of the process flow chart of this invention.

[FIG. 7] It is the general structural figure of the mechanical system of the cryotherapy device provided with the freezing medical apparatus which is the embodiment of this invention.

[FIG. 8] It is the enlarged sectional view of the freezing gas storage means which composes the cryotherapy device of the embodiment of this invention.

[FIG. 9] It is the enlarged perspective view of the gas supply-exhaust pipe connecting the freezing treatment apparatus and the cryotherapy device which is the embodiment of this invention.

[FIG. 10] It is the enlarged sectional view of the freezing treatment apparatus of the embodiment of this invention.

[FIG. 11] It is the enlarged sectional view of the modified example of the freezing treatment apparatus which is the embodiment of this invention.

[FIG. 12] It is the figure of the exhaust system of th embodiment of this invention.

[FIG. 13] It is the example of the animal experimental data.

[FIG. 14] It is the example of the animal experimental data.

[FIG. 15] It is the figure of the example of temperature change, of this invention, by the freezing and the defrosting in the time passage on a plurality of circumferences of the treatment portion.

### EXPLANATION OF NOTATIONS

1 Probe
1a Probe body
1c Evaporation chamber
1d Evaporation means
2 Gas switching means
6 Control means
8 Freezing gas storage means
9 Pressurizing means
10 Defrosting gas supply means
11 Gas supply-exhaust pipe
100 Gas supply-exhaust system
200 Control system
300 Probe system

1. A cryotherapy planning device comprising, in a cryotherapy device wherein a freezing and a defrosting can be performed in a freezing period which freezes a treatment portion and in a defrosting period in which its defrosting is performed, by a freezing probe having a predetermined section size, that said freezing period and said defrosting period are settled according to an organization and a focus treatment size of the treatment portion.

2. A cryotherapy planning device comprising, in a cryotherapy device wherein a freezing and a defrosting can be performed in a freezing period which freezes a treatment portion and in a defrosting period in which its defrosting is performed, that said freezing period and said defrosting period are settled according to a section size of a freezing probe, and an organization and a focus treatment size of the treatment portion.

3. A cryotherapy planning device wherein a relation between a kind of diameter of a freezing probe and a focus treatment size, obtained by a diameter size of the freezing probe and a freezing limit size settled according to the diameter size, is memorized as data, and, by using this data, a freezing probe to be used and a focus treatment size are determined.

4. A cryotherapy planning device wherein a relation between a diameter size of a freezing probe, a freezing limit size settled according to the diameter size and a time width approaching to the freezing limit size, is memorized as data, and, by using this data, a freezing probe to be used, a focus treatment size and a freezing time are determined.

5. A cryotherapy planning device wherein a relation between a diameter size of a freezing probe, a freezing limit size settled according to the diameter size and a time width approaching to the freezing limit size, is memorized as data, and, by using this data, a freezing probe to be used, a focus treatment size, a freezing time, a defrosting period and/or a repetition of cycle of its freezing and defrosting are determined.

6. A cryotherapy planning device according to claim 5, wherein said relation is controlled by a following equation settled by a diameter of a freezing probe, when y is a freezing size and c is a freezing limit size,
y=a exp(bt)+c
wherein exp is an exponential function, a, b and c are coefficients settled by a treatment portion and a diameter of a probe, being in a relation of a<0, b<0, c>0, and c is the freezing limit size.

7. A cryotherapy device comprising a freezing probe; a gas control means to send a freezing gas for freezing a treatment portion during a freezing period and to send a defrosting gas for its defrosting during a defrosting period, to the freezing probe; means for setting a freezing probe to be used, a focus treatment size, a freezing time T1 and a defrosting time T2 by utilizing memorized data of a relation between the diameter size of the freezing probe, a freezing limit size settled according to the diameter size and a time width approaching to the freezing limit size; and commanding means for sending a control command to said gas control means so as to freeze for the settled freezing time T1 and to defrost for defrosting time T2.

8. A cryotherapy device according to claim 7, wherein said relation is controlled by a following equation settled by a diameter of a freezing probe, when y is a freezing size and c is a freezing limit size,
y=a exp(bt)+c
wherein exp is an exponential function, a, b and c are coefficients settled by a treatment portion and a diameter of a probe, being in a relation of a<0, b<0, c>0, and c is the freezing limit size.

9. A freezing treatment period planning device which obtains a freezing period T1 for freezing a treatment portion and a defrosting period T2 to defrost it by utilizing a freezing probe, wherein the freezing period T1 for freezing the treatment portion surrounding the freezing probe by a freezing gas is settled based on a time t obtained by solving an equation of
y=A ln(t)+B (ln is natural logarithm)
wherein A and B are constants settled by an organization of the treatment portion and y is a focus treatment size and the defrosting period T2 is settled based on a time t obtained by solving an equation of
Z=C−D ln(t)
wherein Z is a defrosting size, and C and D are constants settled by an organization of the treatment portion.

10. A cryotherapy planning device according to claim 1, wherein, when a freezing period and a defrosting period are made as 1 cycle, the number of a repetition of the cycle is settled.

11. A cryotherapy planning device according to claim 3, wherein a purge period for purging the freezing gas is added to the freezing period.

12. A cryotherapy device comprising a freezing probe, a gas control means to send a freezing gas for freezing a treatment portion during a freezing period and to send a defrosting gas for its defrosting during a defrosting period, to the freezing probe, means for setting a freezing period T1 which is settled based on a time t obtained by solving an equation of
y=A ln(t)+B (ln is natural logarithm)
wherein A and B are constants settled by an organization of the treatment portion and y is a focus treatment size, and a defrosting period T2 which is settled based on a time t obtained by solving an equation of
Z=C−Dln(t) (ln is natural logarithm)
wherein Z is a defrosting size, and C and D are constants settled by an organization of the treatment portion, and commanding means for sending a control command to said gas control means so as to freeze for the settled freezing period T1 and to defrost for the defrosting period T2.

13. A cryotherapy device according to claim 12, wherein said setting means settle the number of a repetition of cycle, when a freezing period and a defrosting period are made as 1 cycle, and said commanding means send control commands for freezing and defrosting to said gas control means according to the cycle.

14. A cryotherapy device according to claim 12, wherein said setting means add a purge period for purging a freezing gas in case that a liquid freezing gas is utilized to said freezing period, and said commanding means send control commands for freezing, defrosting and purging according to these periods.

15. A cryotherapy device according to claim 14, wherein a gas of a vaporized gas generated in a liquid freezing gas source is used as the purge gas in the purge period.

16. A cryotherapy device according to claim 12, wherein said gas control means has a negative pressure system for exhausting gas.

17. A cryotherapy device according to claim 15, wherein said negative pressure mechanism has a negative monitoring sensor and said gas control means is stopped when said sensor detects a positive pressure turned from a negative pressure.

18. A cryotherapy device according to claim 7, wherein said setting means settle the number of a repetition of cycle, when a freezing period and a defrosting period are made as 1 cycle, and said commanding means send control commands for freezing and defrosting to said gas control means according to the cycle.

19. A cryotherapy device according to claim 7, wherein said setting means add a purge period for purging a freezing gas in case that a liquid freezing gas is utilized to said freezing period, and said commanding means send control commands for freezing, defrosting and purging according to these periods.

20. A cryotherapy therapy device according to claim 7, wherein said gas control means has a negative pressure system for exhausting gas.
